(19) United States
(12) Patent Application Publication
(10) Pub. No.: US 2010/0222624 A1
(43) Pub. Date: Sep. 2, 2010

(54) CATALYST FOR LIQUEFIED PETROLEUM GAS PRODUCTION

(75) Inventors: Kaoru Fujimoto, Fukuoka (JP); Xiaohong Li, Fukuoka (JP)

Correspondence Address: FOLEY AND LARDNER LLP;SUITE 500, 3000 K STREET NW, WASHINGTON, DC, 20007, US

(73) Assignee: JAPAN GAS SYNTHESIZE, LTD., Tokyo (JP)

(21) Appl. No.: 12/279,613

(22) PCT Filed: Feb. 16, 2007
(86) PCT No.: PCT/JP2007/052856

§ 371 Date: Aug. 15, 2008

(30) Foreign Application Priority Data
Feb. 17, 2006 (JP) 2006-041028

(51) Int. Cl. C07C 1/04; B01J 29/068

(52) U.S. Cl. 585/733; 502/74

(57) ABSTRACT

It is an objective of the present invention to provide a catalyst for liquefied petroleum gas production that is less likely to deteriorate over time and is capable of serving as a catalyst in a reaction for producing a liquefied petroleum gas from carbon monoxide and hydrogen under relatively low temperature and pressure conditions.

The present invention relates to a catalyst for producing a liquefied petroleum gas mainly consisting of propane or butane by reacting carbon monoxide with hydrogen, such catalyst comprising a Cu—Zn-based catalyst component and a β-zeolite catalyst component loaded with Pd.

### TECHNICAL FIELD

The present invention relates to a catalyst for producing a liquefied petroleum gas mainly consisting of propane or butane by reacting carbon monoxide with hydrogen.

In addition, the present invention relates to a method for producing a liquefied petroleum gas mainly consisting of propane or butane from a synthesis gas with the use of such catalyst. Further, the present invention relates to a method for producing a liquefied petroleum gas mainly consisting of propane or butane from a carbon-containing starting material such as natural gas with the use of such catalyst.

### BACKGROUND ART

The term “liquefied petroleum gas (LPG)” refers to liquid products obtained by compressing a petroleum-based or natural-gas-based hydrocarbon, which is present in a gas form at ordinary temperatures and pressures, with or without simultaneous cooling. A liquefied petroleum gas mainly contains propane or butane. LPGs that can be stored and transported in liquid form are excellent in terms of portability. Unlike the case of natural gas that needs to be supplied via pipelines, LPGs are characterized in that they can be supplied to any location when loaded into a cylinder. Therefore, an LPG mainly consisting of propane, namely, propane gas, has been widely used as a household/business-use fuel. At present, also in Japan, propane gas is supplied to approximately 25,000,000 households (over 50% of the total households). Further, LPGs have been used not only as household/business-use fuels but also as fuels (mainly containing butane gas) for portable devices or apparatuses such as portable gas stoves or disposable lighters, industrial-use fuels, and automobile fuels.

Hitherto, LPGs have been produced by the following methods: 1): a method for recovering an LPG from a wet natural gas; 2): a method for recovering an LPG in a step of stabilizing crude oil (with vapor pressure control); and 3): a method for separating/extracting a product generated in a petroleum refining step.

LPGs, and in particular, propane gas used as a household/business-use fuel, are expected to be in demand in the future. Thus, the establishment of a novel method for producing an LPG in an industrially practicable manner is extremely useful.

Patent Document 1 discloses an LPG production method for producing a liquefied petroleum gas or a hydrocarbon mixture having a composition similar to that of a liquefied petroleum gas by reacting a synthesis gas comprising hydrogen and carbon monoxide in the presence of, for example, a Cu—Zn-based, Cr—Zn-based, or Pd-based methanol synthesis catalyst, and specifically, a mixed catalyst obtained by physically mixing a CuO—ZnO—Al₂O₃ catalyst, a Pd/SiO₂ catalyst, and a methanol conversion catalyst comprising zeolite, such as Y-type zeolite, having an average pore size of approximately 10 angstroms (1 nm) or more.

Patent Document 1 describes, with reference to a zeolite catalyst, that the distribution of a generated hydrocarbon strongly depends on zeolite pore size, and thus the generation of an aromatic hydrocarbon can be suppressed with the use of zeolite (Y-type zeolite) having a large pore size and C1-C6 lower paraffin, and in particular, C2-C4 lower paraffin, can be synthesized at a high selection rate. Also, the above Patent Document 1 describes that any zeolite catalyst can be used, regardless of molecular structure or pore structure variations and the use or nonuse of different preparation treatments as long as the above conditions are satisfied, although the pore size is limited. Meanwhile, Patent Document 1 describes, with reference to a methanol synthesis catalyst, that a simple substance or complex of a different metal or metallic oxide can be used as a methanol synthesis catalyst as long as it has hydrogenation ability.

Further, Patent Document 1 describes that the yield of a hydrocarbon (lower paraffin) is improved by increasing the acidity of zeolite by dealuminization treatment based on comparison of the results of a reaction using a mixed catalyst comprising, as a methanol conversion catalyst, dealuminized Y-type zeolite (SiO₂/Al₂O₃=7.6) and the results of a reaction using a mixed catalyst comprising, as a methanol conversion catalyst, Y-type zeolite (SiO₂/Al₂O₃=5.1) not subjected to dealuminization treatment.

However, it cannot necessarily be said that catalysts described in Patent Document 1 have satisfactory properties.

For instance, a catalyst comprising Pd/SiO₂ and Y-type zeolite has low activity and hydrocarbon yield. Also, in such case, the contents of propane (C3) and butane (C4) in generated hydrocarbon become low. A catalyst comprising Pd/SiO₂ and a product obtained by subjecting dealuminized Y-type zeolite (SiO₂/Al₂O₃=7.6) to water vapor treatment at 450° C. for 2 hours has relatively high activity and hydrocarbon yield. Also, in such case, the contents of propane (C3) and butane (C4) in generated hydrocarbon become relatively high. However, it is still difficult to say that such catalyst has sufficiently excellent properties, particularly in terms of activity and hydrocarbon yield. Further, a catalyst comprising Pd/SiO₂ and Y-type zeolite generally deteriorates over time to a significant extent. Thus, it is difficult to say that the catalyst life is sufficiently long.

Meanwhile, a Cu—Zn-based catalyst (a methanol synthesis catalyst comprising a copper-zinc-alumina mixed oxide) and a catalyst comprising Y-type zeolite generally tend to have higher activity and hydrocarbon yield than a catalyst comprising Pd/SiO₂ and Y-type zeolite. Also, in such cases, the contents of propane (C3) and butane (C4) in generated hydrocarbon become high. In particular, a catalyst comprising a Cu—Zn-based catalyst and a product obtained by subjecting dealuminized Y-type zeolite (SiO₂/Al₂O₃=7.6) to water vapor treatment at 450° C. for 2 hours has high activity and hydrocarbon yield. Also, in such case, the contents of propane (C3) and butane (C4) in generated hydrocarbon become high. However, in general, a catalyst comprising a Cu—Zn-based catalyst and Y-type zeolite deteriorates over time to a significant extent. Thus, it is difficult to say that the catalyst life is sufficiently long. Therefore, it is difficult to produce an LPG at a high yield with the use of such catalyst for a long period of time in a stable manner.

As described above, further improvement of catalysts for liquefied petroleum gas production has been awaited in order to practically realize a process for LPG production from a synthesis gas, and further, a process for LPG production from a carbon-containing starting material such as natural gas.

In addition, a catalyst comprising a Cr—Zn-based catalyst and a β-zeolite has been examined as a catalyst for liquefied petroleum gas production. However, a reaction temperature of as high as approximately 400° C. is necessary for such catalyst, which is problematic.

In general, a reaction represented by the following formula (I) takes place by reacting carbon monoxide with hydrogen in the presence of a catalyst for liquefied petroleum gas production that comprises a methanol synthesis catalyst component and a zeolite catalyst component. Thus, an LPG mainly consisting of propane or butane can be produced.

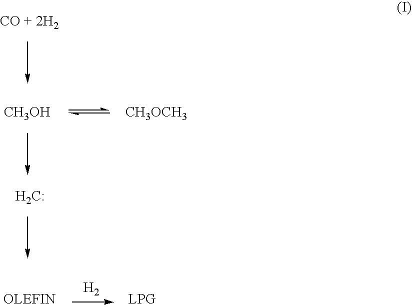

At first, methanol is synthesized from carbon monoxide and hydrogen on a methanol synthesis catalyst component. At such time, dimethyl ether is also generated as a result of dehydrodimerization of methanol. Next, the thus synthesized methanol is converted into a lower olefin hydrocarbon mainly containing propylene or butene at active sites in pores of a zeolite catalyst component. In such reaction, it is considered that carbene (H₂C:) is generated as a result of dehydration of methanol and then lower olefin is generated as a result of polymerization of the obtained carbene. Then, the generated lower olefin is released from pores in the zeolite catalyst component and immediately hydrogenated on a methanol synthesis catalyst component. Accordingly, paraffin mainly containing propane or butane (namely, LPG) is obtained.

The term “methanol synthesis catalyst component” used herein refers to a component that exhibits catalyst actions in the reaction represented by CO+2H₂→CH₃OH. In addition, the term “zeolite catalyst component” refers to zeolite that exhibits catalyst actions in a methanol-to-hydrocarbon condensation reaction and/or a dimethyl ether-to-hydrocarbon condensation reaction.

### Patent Document 1: JP Patent Publication (Kokai) No. 61-23688 A (1986)

### DISCLOSURE OF THE INVENTION

### Problem to be solved by the Invention

It is an objective of the present invention to provide a catalyst for liquefied petroleum gas production that is capable of serving as a catalyst in a reaction for producing a liquefied petroleum gas from carbon monoxide and hydrogen under relatively low temperature and pressure conditions.

It is another objective of the present invention to provide a catalyst for liquefied petroleum gas production that is less likely to deteriorate over time and can be stably used for many hours.

### Means for Solving the Problem

The present application includes the following inventions.

[1] A catalyst for producing a liquefied petroleum gas mainly consisting of propane or butane by reacting carbon monoxide with hydrogen, the catalyst comprising a Cu—Zn-based catalyst component and a β-zeolite catalyst component loaded with Pd.
[2] The catalyst according to [1], wherein the loaded Pd content in the β-zeolite catalyst component loaded with Pd is 0.1% to 1% by weight.
[3] The catalyst according to [1] or [2], wherein β-zeolite in the β-zeolite catalyst component loaded with Pd is (β-zeolite having an SiO₂:Al₂O₃ molar ratio of 10:1 to 150:1.
[4] The catalyst according to any one of [1] to [3], wherein the weight ratio of the Cu—Zn-based catalyst component to the β-zeolite catalyst component loaded with Pd is 4:1 to 1:4.
[5] The catalyst according to any one of [1] to [4], wherein the β-zeolite catalyst component loaded with Pd is prepared by a method comprising the steps of immersing β-zeolite in a solution containing Pd(NH₃)₄Cl₂ and removing Cl from the β-zeolite treated in the previous step.
[6] A method for liquefied petroleum gas production, comprising reacting carbon monoxide with hydrogen in the presence of the catalyst according to any one of [1] to [5] so as to produce a liquefied petroleum gas mainly consisting of propane or butane.
[7] The method according to [6], wherein the reaction temperature for reacting carbon monoxide with hydrogen is 290° C. to 375° C.
[8] The method according to [6] or [7], wherein the reaction pressure for reacting carbon monoxide with hydrogen is 2 to 5 MPa.
[9] The method according to any one of [6] to [8], wherein a value obtained by dividing the amount of a catalyst used for reacting carbon monoxide with hydrogen (units: “g”) by the inlet gas flow rate (units: “mol/h”) is 1.9 to 18 g·h/mol.
[10] A method for liquefied petroleum gas production, comprising a liquefied petroleum gas production step of allowing a synthesis gas to flow through a catalyst layer containing the catalyst according to any one of [1] to [5] so as to produce a liquefied petroleum gas mainly consisting of propane or butane.
[11] A method for liquefied petroleum gas production, comprising:

(1): a step of producing a synthesis gas with the use of a carbon-containing starting material and at least one member selected from the group consisting of H₂O, O₂, and CO₂; and

(2): a liquefied petroleum gas production step of allowing a synthesis gas to flow through a catalyst layer containing the catalyst according to any one of [1] to [5] so as to produce a liquefied petroleum gas mainly consisting of propane or butane.

### EFFECTS OF THE INVENTION

According to the present invention, the following are provided: a catalyst for liquefied petroleum gas production that is less likely to deteriorate over time and is capable of serving as a catalyst in a reaction for producing a liquefied petroleum gas from carbon monoxide and hydrogen under relatively low temperature and pressure conditions; and a method for liquefied petroleum gas production with the use of the same.

This description includes part or all of the contents as disclosed in the description and/or drawings of Japanese Patent Application No. 2006-41028, which is a priority document of the present application.

### BRIEF DESCRIPTION OF THE DRAWINGS

FIG. 1A shows CO conversion rates and C3+C4 selection rates obtained 3 hours after the initiation of the flow of a starting material gas in LPG synthesis reactions using a variety of catalysts each having a different SiO₂:Al₂O₃ molar ratio.

FIG. 1B shows hydrocarbon compositions of products obtained 3 hours after the initiation of the flow of a starting material gas in LPG synthesis reactions using a variety of catalysts each having a different SiO₂:Al₂O₃ molar ratio.

FIG. 2A shows CO conversion rates and C3+C4 selection rates obtained 3 hours after the initiation of the flow of a starting material gas in the cases of catalyst beds having different lengths.

FIG. 2B shows hydrocarbon compositions of products obtained 3 hours after the initiation of the flow of a starting material gas in the cases of catalyst beds having different lengths.

FIG. 3A shows CO conversion rates and C3+C4 selection rates obtained 3 hours after the initiation of the flow of a starting material gas at different reaction temperatures.

FIG. 3B shows hydrocarbon compositions of products obtained 3 hours after the initiation of the flow of a starting material gas at different reaction temperatures.

FIG. 4A shows CO conversion rates and C3+C4 selection rates obtained 3 hours after the initiation of the flow of a starting material gas at different reaction pressures.

FIG. 4B shows hydrocarbon compositions of products obtained 3 hours after the initiation of the flow of a starting material gas at different reaction pressures.

FIG. 5A shows CO conversion rates and C3+C4 selection rates obtained 3 hours after the initiation of the flow of a starting material gas at different W/F values.

FIG. 5B shows hydrocarbon compositions of products obtained 3 hours after the initiation of the flow of a starting material gas at different W/F values.

FIG. 6 shows time-dependent changes in the CO conversion rate in LPG synthesis reactions using a variety of β-zeolite catalysts having different loaded Pd contents.

FIG. 7 shows time-dependent changes in the C3+C4 selection rate in LPG synthesis reactions using a variety of β-zeolite catalysts having different loaded Pd contents.

FIG. 8 shows time-dependent changes in the hydrocarbon composition of a product in an LPG synthesis reaction using a non-Pd-loaded β-zeolite catalyst.

FIG. 9 shows time-dependent changes in the hydrocarbon composition of a product in an LPG synthesis reaction using a β-zeolite catalyst loaded with Pd (0.17% by weight).

FIG. 10 shows time-dependent changes in the hydrocarbon composition of a product in an LPG synthesis reaction using a β-zeolite catalyst loaded with Pd (0.5% by weight).

FIG. 11 shows time-dependent changes in the hydrocarbon composition of a product in an LPG synthesis reaction using a β-zeolite catalyst loaded with Pd (1% by weight).

FIG. 12 shows time-dependent changes in the yields of CO₂, dimethyl ether (DME), and hydrocarbon of a product in an LPG synthesis reaction using a β-zeolite catalyst loaded with Pd (1% by weight).

FIG. 13 shows in more detail time-dependent changes in the hydrocarbon composition of a product in an LPG synthesis reaction using a β-zeolite catalyst loaded with Pd (0.5% by weight).

FIG. 14A shows time-dependent changes in the yields of CO₂, dimethyl ether (DME), and hydrocarbon of a product in an LPG synthesis reaction using a β-zeolite catalyst loaded with Pd by an ion exchange method.

FIG. 14B shows in more detail time-dependent changes in the hydrocarbon composition of a product in an LPG synthesis reaction using a β-zeolite catalyst loaded with Pd by an ion exchange method.

FIG. 15A shows time-dependent changes in the yields of CO₂, dimethyl ether (DME), and hydrocarbon of a product in an LPG synthesis reaction using a β-zeolite catalyst loaded with Pd by an immersion method.

FIG. 15B shows in more detail time-dependent changes in the hydrocarbon composition of a product in an LPG synthesis reaction using a β-zeolite catalyst loaded with Pd by an immersion method.

FIG. 16A shows the CO conversion rate (%), the hydrocarbon yield (%), and the LPG selectivity (C3+C4 selectivity) (%) at each time point after the initiation of the LPG synthesis reaction in Example 2 with the use of the catalyst of the present invention.

FIG. 16B shows the product composition with regard to each hydrocarbon (C %) at each time point after the initiation of the LPG synthesis reaction in Example 2 with the use of the catalyst of the present invention.

### BEST MODE FOR CARRYING OUT THE INVENTION

### Cu—Zn-Based Catalyst Component

In the present invention, a Cu—Zn-based catalyst component has functions of a methanol synthesis catalyst and of an olefin hydrogenation catalyst. The term “Cu—Zn-based catalyst” refers to a catalyst mainly containing a composite oxide comprising copper and zinc. Typical examples thereof include a catalyst mainly consisting of a copper-zinc-alumina mixed oxide.

Any Cu—Zn-based catalyst component may be used as long as it has functions of a methanol synthesis catalyst and of an olefin hydrogenation catalyst. A commercially available product (manufactured by, for example, Nippon Kokan K.K.) can be used as a Cu—Zn-based catalyst.

(β-Zeolite Catalyst Component Loaded with Pd)

According to the present invention, the β-zeolite catalyst component loaded with Pd exhibits catalytic action during a methanol-to-hydrocarbon condensation reaction and/or a dimethyl ether-to-hydrocarbon condensation reaction. According to the present invention, any β-zeolite catalyst component may be used as long as it is loaded with Pd and has catalytic actions.

The term “β-zeolite catalyst component loaded with Pd” used herein is sometimes replaced with “Pd-loaded β-zeolite catalyst component.”

In addition, each pore in β-zeolite is formed with a 12-membered oxygen ring. The pore size is approximately 0.66×0.76 nm. β-zeolite before being subjected to Pd loading is preferably high-silica β-zeolite. Specifically, β-zeolite having an SiO₂:Al₂O₃ molar ratio of 10:1 to 150:1 is preferable. With the use of β-zeolite having an SiO₂:Al₂O₃ molar ratio of 10:1 to 150:1, it is possible to convert generated methanol to olefin mainly consisting of propylene or butene and further to a liquefied petroleum gas mainly consisting of propane or butane in a more selective manner. β-zeolite has an SiO₂:Al₂O₃ molar ratio of more preferably 20:1 to 100:1 and most preferably 30:1 to 50:1. Commercially available proton-type β-zeolite can be used as the above β-zeolite.

The present invention is characterized in that β-zeolite is loaded with Pd. Preferably, the loaded Pd content in a β-zeolite catalyst component is 0.1% by weight or more. The upper limit of such content is not particularly limited. However, it is generally 1% by weight or less, more preferably 0.5% by weight or less, and most preferably 0.2% by weight or less. In addition, the loaded Pd content (% by weight) is defined as follows.

The loaded Pd content(% by weight)=[(Pd weight)/(Pd weight+β-zeolite weight)]×100

It is possible to subject β-zeolite to Pd loading by, for example, immersing β-zeolite powder in a solution containing Pd, taking the powder out of the solution after the elapse of a certain period of time, and drying the powder. Surprisingly, the present inventors have found that not only the yield of hydrocarbon but also the LPG selectivity can be improved upon LPG synthesis reaction in a case in which Pd loading is carried out by a method comprising the steps of immersing β-zeolite in a solution containing Pd(NH₃)₄Cl₂ and removing Cl from the β-zeolite by washing the β-zeolite treated in the previous step with ion-exchange water (an ion exchange method), compared with a case in which Pd loading is carried out by a method comprising a step of immersing β-zeolite in a solution containing Pd(NH₃)₄Cl₂ (an immersion method). That is, the Pd-loaded β-zeolite catalyst component of the present invention is preferably produced by a method comprising the steps of immersing β-zeolite in a solution containing Pd(NH₃)₄Cl₂ and removing Cl from the β-zeolite treated in the previous step.

### (A Method for Producing a Catalyst for Liquefied Petroleum Gas Production)

In the method for producing a catalyst for liquefied petroleum gas production of the present invention, it is preferable to separately prepare a Cu—Zn-based catalyst component and a Pd-loaded β-zeolite catalyst component and to mix them. In view of functions of each component, it is possible to easily and optimally design the composition, the structure, and the physical properties thereof by separately preparing a Cu—Zn-based catalyst component and a Pd-loaded β-zeolite catalyst component.

The mixing ratio of a Cu—Zn-based catalyst component to a Pd-loaded β-zeolite catalyst component is not particularly limited. However, [the weight of Cu—Zn-based catalyst component]: [the weight of β-zeolite catalyst component loaded with Pd] is preferably 4:1 to 1:4 and more preferably 2:1 to 1:2.

It is possible to separately subject a Cu—Zn-based catalyst component and a Pd-loaded β-zeolite catalyst component to molding and to mix them for use. The mixture obtained by such mixing may be further subjected to molding. In addition, it is possible to subject a mixed powder to molding after mixing a Cu—Zn-based catalyst component powder and a Pd-loaded β-zeolite catalyst component powder.

A method for mixing/molding both catalyst components is not particularly limited. However, mixing/molding is preferably carried out by a dry method. When mixing/molding of both catalyst components is carried out by a wet method, a compound is transferred between both catalyst components. Accordingly, physical properties of both components, which are optimized in accordance with their functions, might vary. Examples of a catalyst molding method include an extrusion molding method and a molding method involving tablet making.

In the present invention, it is preferable that a Cu—Zn-based catalyst component and a Pd-loaded β-zeolite catalyst component to be mixed have somewhat large particle sizes. Both components may be formed into powder or granules. Preferably, they are formed into granules.

The term “powder” herein used refers to powder having an average particle size of 10 μm or less. The term “granule” refers to granule having an average particle size of 100 μm or more.

Preferably, a Cu—Zn-based catalyst component and a Pd-loaded β-zeolite catalyst component to be mixed have the same average particle size.

A Cu—Zn-based catalyst component in a granule form (i.e., with an average particle size of 100 μm or more) and a Pd-loaded β-zeolite catalyst component in a granule form (i.e., with an average particle size of 100 μm or more) are mixed with each other and subjected to molding according to need such that the catalyst for liquefied petroleum gas production of the present invention is produced. Thus, it becomes possible to obtain a catalyst that has a prolonged catalyst life and is less likely to deteriorate. The average particle size of a Cu—Zn-based catalyst component and the average particle size of a Pd-loaded β-zeolite catalyst component to be mixed are preferably 200 μm or more and more preferably 500 μm or more.

Meanwhile, in order to retain the excellent properties of the mixed catalyst of the present invention, the average particle sizes of a Cu—Zn-based catalyst component and a Pd-loaded β-zeolite catalyst component to be mixed are preferably 5 mm or less and more preferably 2 mm or less.

In general, when the catalyst for liquefied petroleum gas production of the present invention is produced by mixing a Cu—Zn-based catalyst component in a granule form and a Pd-loaded β-zeolite catalyst component in a granule form, each catalyst component is previously subjected to a conventional molding method such as a molding method involving tablet making or an extrusion molding method, each resultant is disrupted in a mechanical manner according to need, and the average particle size of each resultant is adjusted to preferably approximately 100 μm to 5 mm, followed by mixing in a uniform manner. Then, the obtained mixture is further subjected to molding according to need such that the catalyst for liquefied petroleum gas production of the present invention is produced.

On the other hand, when the catalyst for liquefied petroleum gas production of the present invention is produced by mixing a Cu—Zn-based catalyst component in a powder form and a Pd-loaded β-zeolite catalyst component in a powder form, each catalyst component is generally disrupted in a mechanical manner according to need, and the average particle size of each resultant is adjusted to, for example, approximately 0.5 to 2 μm, followed by mixing in a uniform manner and molding according to need. Alternatively, all desired catalyst components are added and mixed together, mixing is carried out with disruption in a mechanical manner until the resultant is uniformly mixed, and the average particle size of the resultant is adjusted to, for example, approximately 0.5 to 2 μm, followed by molding according to need.

In addition, the catalyst for liquefied petroleum gas production of the present invention may contain other components to be added according to need, unless the desired effects thereof are lost. For instance, for the purpose of temperature control, it is possible to use the catalyst for liquefied petroleum gas production of the present invention diluted with silica, alumina, or an inert and stable heat conductor. Further, for the purpose of temperature control, it is possible to use the catalyst for liquefied petroleum gas production of the present invention applied to the surface of a heat exchanger. In addition, when an inert component such as silica is added to the catalyst, the total volume of the catalyst is increased. Accordingly, the length of a catalyst bed obtained by filling a column with the catalyst becomes longer and thus the CO conversion rate and the LPG selection rate can be improved.

Moreover, when it is necessary to activate a catalyst component to be used by reduction treatment before use, such catalyst component may be activated at such prior stage by reduction treatment. Alternatively, the catalyst for liquefied petroleum gas production of the present invention is produced by mixing/molding a Cu—Zn-based catalyst component and a Pd-loaded β-zeolite catalyst component. Then, the catalyst components may be activated by reduction treatment before the initiation of reaction. Conditions for reduction treatment are not particularly limited.

### (A Method for Liquefied Petroleum Gas Production)

Next, a method for producing a liquefied petroleum gas mainly consisting of propane or butane, and preferably, propane, by reacting carbon monoxide with hydrogen with the use of the aforementioned catalyst for liquefied petroleum gas production of the present invention is described.

The reaction temperature is preferably 290° C. to 375° C. and more preferably 320° C. to 350° C. When the reaction temperature falls within the above range, it is possible to produce propane and/or butane at a higher conversion rate and a higher yield. As described above, it has become possible to carry out an LPG synthesis reaction under relatively low temperature conditions with the use of the catalyst of the present invention.

The reaction pressure is preferably 2 to 5 MPa and more preferably 3 to 4 MPa. When the reaction pressure is low, the CO conversion rate tends to decrease. In addition, when the pressure is excessively high, a hydrocarbon with 5 or more carbons, which is not an LPG component, tends to be generated.

The value (W/F value) obtained by dividing the amount of catalyst used for reacting carbon monoxide with hydrogen (W) (units: “g”) by the inlet gas flow rate (F) (units: “mol/h”) is preferably 1.9 to 18 g·h/mol and more preferably 4 to 10 g·h/mol. When the W/F value is low, the CO conversion rate tends to decrease. In addition, when the W/F value is excessively high, undesirable C1 and C2 hydrocarbons tend to be generated.

The carbon monoxide concentration of a gas that is introduced into a reactor is preferably 20 mol % or more and more preferably 25 mol % or more from the viewpoints of the securement of the pressure (partial pressure) of carbon monoxide necessary for reaction and of the improvement of the starting material unit consumption. In addition, the carbon monoxide concentration of a gas that is introduced into a reactor is preferably 45 mol % or less and more preferably 40 mol % or less from the viewpoint of a more sufficient increase in the carbon monoxide conversion rate.

The hydrogen concentration of a gas that is introduced into a reactor is preferably 1.2 mol or more and more preferably 1.5 mol or more with respect to 1 mol of carbon monoxide from the viewpoint of more sufficient reaction of carbon monoxide. In addition, the hydrogen concentration of a gas that is introduced into a reactor is preferably 3 mol or less and more preferably 2.5 mol or less with respect to 1 mol of carbon monoxide from the viewpoint of economic efficiency. In addition, if necessary, it is also preferable to reduce the hydrogen concentration of a gas that is introduced into a reactor to approximately 0.5 mol with respect to 1 mol of carbon monoxide.

A gas that is introduced into a reactor may be obtained by adding carbon dioxide to carbon monoxide and hydrogen serving as reaction starting materials. It is possible to substantially reduce carbon dioxide production from carbon monoxide as a result of shift reaction in a reactor by recycling carbon dioxide emitted from the reactor or adding carbon dioxide in an amount corresponding to the amount of emitted carbon dioxide. Further, it is also possible to offset the carbon dioxide production.

In addition, a gas that is introduced into a reactor may contain water vapor. In addition to the above, a gas that is introduced into a reactor may contain an inert gas or the like.

Also, a gas that is introduced into a reactor is portioned and then introduced into a reactor such that it is possible to control the reaction temperature.

The reaction can be carried out with the use of a fixed bed, a fluidized bed, or a moving bed. Such bed is preferably selected in view of both reaction temperature control and catalyst reproduction method. For instance, examples of a fixed bed reactor that can be used include a quench-type reactor employing an internal multiple quench system, a multitubular reactor, a multiple reactor accommodating a plurality of heat exchangers, and other reactors employing a multiple cooling radial flow system, a double-tube heat exchange system, a built-in cooling coil system, a mixed flow system, and the like.

(A Method for Producing a Liquefied Petroleum Gas from a Carbon-Containing Starting Material)

According to the LPG production method of the present invention, a synthesis gas that is used as a starting material gas can be produced with a carbon-containing starting material and at least one member selected from the group consisting of H₂O, O₂, and CO₂.

A carbon-containing starting material that can be used is a carbon-containing substance capable of producing H₂ and CO as a result of a reaction with at least one member selected from the group consisting of H₂O, O₂, and CO₂. As a carbon-containing starting material, a known starting material for a synthesis gas can be used. Examples of such starting material that can be used include a lower hydrocarbon such as methane or ethane, natural gas, naphtha, and coal.

In the present invention, a catalyst is used in a liquefied petroleum gas production step. Therefore, a carbon-containing starting material (e.g., natural gas, naphtha, or coal) preferably has a low content of a catalyst-poisoning substance such as sulfur, a sulfur compound, or the like. In addition, when a carbon-containing starting material contains a catalyst-poisoning substance, it is possible to carry out a step of removing the catalyst-poisoning substance (involving desulfurization or the like) before a synthesis gas production step, according to need.

A synthesis gas is produced by reacting a carbon-containing starting material as described above with at least one member selected from the group consisting of H₂O, O₂, and CO₂ in the presence of a catalyst for synthesis gas production (reforming catalyst).

A synthesis gas can be produced by a conventional method. For instance, when natural gas (methane) is employed as a starting material, a synthesis gas can be produced by a water vapor reforming method or an auto-thermal reforming method. In such case, it is possible to supply water vapor necessary for water vapor reforming, oxygen necessary for auto-thermal reforming, or the like according to need. In addition, in a case in which coal is designated as a starting material, a synthesis gas can be produced using an air-blast gasification furnace or the like.

Also, a shift reactor, for example, may be installed downstream of a reformer serving as a reactor for producing a synthesis gas from a starting material as described above such that the composition of the synthesis gas can be controlled by shift reaction (CO+H₂O→CO₂+H₂).

The present invention is hereafter described in greater detail with reference to the following examples, although the technical scope of the present invention is not limited thereto.

### Example 1

### 1. Catalysts

### (Cu—Zn Catalyst Component)

A Cu—Zn catalyst (Nippon Kokan K.K.) was used as a methanol synthesis catalyst.

### (Pd-Loaded β-Zeolite Catalyst Component)

A Pd-loaded β-zeolite catalyst component used was obtained by loading a commercially available β-zeolite catalyst with Pd ions.

The commercially available β-zeolite catalyst used was a proton-type β-zeolite having an SiO₂:Al₂O₃ molar ratio of 27.5:1 (Catalysts & Chemicals Ind. Co., Ltd.) or a proton-type β-zeolite product having an SiO₂:Al₂O₃ molar ratio of 37.1:1, 50:1, 75:1, 100:1, or 150:1 (Tosoh Corporation).

A commercially available β-zeolite catalyst was subjected to Pd loading by the following method.

### (Pd Loading by an Ion Exchange Method)

In this Example, unless otherwise specified, a commercially available β-zeolite product was subjected to Pd loading by an ion exchange method described in this paragraph. An example case in which the loaded Pd content was 0.5% by weight is described. Pd(NH₃)₄Cl₂ (6 mg of Pd/ml) was prepared and diluted to 100 ml. The volume of the Pd(NH₃)₄Cl₂ solution corresponding to a loaded Pd content of 0.5% by weight was calculated. The Pd(NH₃)₄Cl₂ solution in the calculated volume was placed in a container. Ion-exchange water was added to the container to a volume of 25 ml. A β-zeolite catalyst (3 g) was added to 25 ml of the solution and suspended therein. Stirring was carried out at 60° C. to 70° C. for 8 hours. The suspension was filtered, followed by washing with ion-exchange water until no Cl ions were found in the filtrate (by checking Cl ions in the filtrate with silver nitrate). The catalyst was placed in a dryer, followed by drying at 120° C. for 12 hours. Then, the resultant was placed in a catalyst sintering machine, followed by sintering at 500° C. for 2 hours. The catalyst was obtained in a powder form. Catalyst preparation was carried out in the same manner also in the cases of the loaded Pd contents of 0.17% by weight and 1% by weight.

### (Pd Loading by an Immersion Method)

In Experiment 7, loading on β-zeolite was carried out by an immersion method. An example case in which the loaded Pd content was 0.5% by weight is described.

The volume of the Pd(NH₃)₄Cl₂ solution corresponding to a loaded Pd content of 0.5% by weight was calculated. The Pd(NH₃)₄Cl₂ solution in the calculated volume was placed in a container. Ion-exchange water was added thereto to a volume of 3 ml based on the constant volume method. A β-zeolite catalyst (3 g) was added thereto. The catalyst was placed in a dryer, followed by drying at 120° C. for 24 hours and sintering at 500° C. for 2 hours. The catalyst was obtained in a powder form.

### (Mixing of the Catalysts)

The Cu—Zn catalyst in a powder form was subjected to pressure molding at 40 kg/cm² for 30 seconds with the use of a tablet-molding machine and then was disrupted into 0.37- to 0.84-mm particles.

Also, the prepared Pd-loaded β-zeolite catalyst in a powder form was subjected to pressure molding at 40 kg/cm² for 30 seconds with the use of a tablet-molding machine and then was disrupted into 0.37- to 0.84-mm particles.

The above two catalyst components that had been separately prepared were mixed together at a certain weight ratio.

This reaction system is for an exothermic reaction. Since uniform temperature distribution was observed in a catalyst layer, silica, which is an inactive substance, was used for dilution in some experiments. Silica Q3 (Tosoh Corporation) was used as silica. The particle size of silica Q3 was 75 to 500 μm, and silica Q3 was mixed with the other two components without disruption.

### 2. Pretreatment of the Catalyst

The catalyst loaded into a reactor was dried with a flow of high purity N₂ at 100 ml/min at 250° C. for 2 hours before reaction. Then, the catalyst was subjected to reduction treatment with a flow of high purity H₂/N₂ (=5/95) at 100 ml/min at 300° C. for 3 hours.

### 3. Gas Used for Reaction

A mixed gas (CO: CO₂: H₂: Ar=24:8:65:3 (molar ratio)) was used as a starting material gas for LPG synthesis reaction.

### 4. Reactor

A pressurized fixed-bed flow reactor was used for reaction. A reaction tube made of stainless steel (internal diameter: 6 mm; total length: 30 cm) was used. The inside of the reaction tube was filled with glass wool, glass beads, a catalyst, and glass beads in such order. The reaction tube was placed in an electric furnace. The temperature of the electric furnace was measured with a thermocouple inserted into a center portion of the furnace under PID control. The temperature of the catalyst was measured with a thermocouple inserted into a catalyst layer in the reaction tube.

### 5. Reaction Conditions

Different conditions of reaction temperature, reaction pressure, catalyst amount, reaction gas flow rate, W/F, loaded Pd content, and weight ratio of a methanol catalyst to zeolite were determined.

### 6. Gas Composition Analysis

After the elapse of a certain period of time from initiation of reaction, gas analysis was carried out with the use of an online-connected gas chromatograph. The gas chromatograph used was GC-8A (Shimadzu Corporation). Table 1 shows analytes and analysis conditions.

TABLE 1

| | | Column | | | | |
|---|---|---|---|---|---|---|
| Analyte | Detector | Filler | Length | Temperature | Carrier | Apparatus |
| Hydrocarbon, DME, CH₃OH | FID | Propack-Q | 4.0 m | 60° C. to 230° C. | He | GC-8A |
| | | | | (5° C./min) | 26 ml/min | |
| CO, CO₂, CH₄, Ar | TCD | Activated carbon | 2.0 m | 100° C. | Ar | GC-8A |
| | | | | | 82 ml/min | |

### 7. Experimental Operational Procedures

An example of experimental procedures (reaction pressure: 21 MPa; reaction temperature: 350° C.) is described below.

(1) A reaction tube is filled with glass wool, glass beads, a catalyst, and glass beads in such order and placed in a furnace.
(2) Leakage from a reactor is checked with a flow of N₂ at 100 ml/min.
(3) A flow of N₂ at 100 ml/min is supplied to the reactor and the temperature is increased to 250° C.
(4) When the temperature reaches 250° C., drying is carried out for 2 hr while the temperature is maintained.
(5) A flow of high purity H₂ at 5 ml/min is supplied with the flow of N₂, the flow of N₂ is set at 95 ml/min, and the temperature is increased to 300° C.
(6) When the temperature reaches 300° C., reduction is carried out for 3 hr while the temperature is maintained.
(7) The supply of N₂ and H₂ is terminated, a mixed gas (CO: CO₂: H₂: Ar=24:8:65:3 (molar ratio)) is supplied, and the pressure is increased to 2.1 MPa.
(8) The temperature is increased to 350° C.
(9) Reaction is initiated.
(10) Sampling is carried out at regular time intervals and the generated gas is analyzed.
(11) The supply of the reaction gas is terminated, the reaction is completed, and the temperature is decreased in the presence of a flow of the reaction gas.

### 8. Definition of Measurement Values

In the following experiment, mainly, “CO conversion rate (%),” “C3+C4 selection rate (%),” and “hydrocarbon composition (C %)” were calculated as indexes for evaluation of catalysts and reaction conditions. Such indexes are defined as follows.

The term “CO conversion rate” refers to the percentage of CO (in a reaction starting material gas) converted to a hydrocarbon and the like.

CO conversion rate(%)=[(inlet CO flow rate(mol/h)−outlet CO flow rate(mol/h))/inlet CO flow rate(mol/h)]×100

The term “C3+C4 selection rate” refers to the content of C3+C4 as a portion of all generated hydrocarbons in terms of carbon.

C3+C4 selection rate(%)=[(C3 generation rate×3+C4 generation rate×4)/(C1 generation rate×1+C2 generation rate×2+C3 generation rate×3+C4 generation rate×4+C5 generation rate×5+C6 generation rate×6 . . . )]×100

The units for generation rate used herein are “mol/h” in each case.

The term “hydrocarbon composition (C %)” refers to the content of an individual hydrocarbon as a portion of all generated hydrocarbons in terms of carbon. For instance, the content of C5 among all hydrocarbons is calculated as follows.

C5 among all hydrocarbons(%)=[(C5 generation rate×5)/(C1 generation rate×1+C2 generation rate×2+C3 generation rate×3+C4 generation rate×4+C5 generation rate×5+C6 generation rate×6 . . . )]×100

The units for generation rate used herein are “mol/h” in each case.

### 9. Experimental Results

### Experiment 1

In this experiment, β-zeolite was examined in terms of the relationship between the SiO₂:Al₂O₃ molar ratio and the CO conversion rate (%), the C3+C4 selection rate (%), or the hydrocarbon composition (C %) of a product.

Reaction conditions are as follows.

TABLE 2

| | Condition 1 | Condition 2 | Condition 3 | Condition 4 | Condition 5 | Condition 6 |
|---|---|---|---|---|---|---|
| Catalyst | 2:1:0 | 2:1:0 | 2:1:0 | 2:1:0 | 2:1:0 | 2:1:0 |
| Cu—Zn:Pd- | | | | | | |
| β zeolite:Silica | | | | | | |
| Q3 | | | | | | |
| (inactive | | | | | | |
| component) | | | | | | |
| (weight ratio) | | | | | | |
| Loaded Pd | 0.5 | 0.5 | 0.5 | 0.5 | 0.5 | 0.5 |
| content in Pd-β | | | | | | |
| zeolite (% by | | | | | | |
| weight) | | | | | | |
| SiO₂:Al₂O₃ | 27.5:1 | 37.1:1 | 50:1 | 75:1 | 100:1 | 150:1 |
| (molar ratio) in | | | | | | |
| β-zeolite | | | | | | |
| Catalyst | 1 | 1 | 1 | 1 | 1 | 1 |
| weight (g) | | | | | | |
| Reaction | 350 | 350 | 350 | 350 | 350 | 350 |
| temperature | | | | | | |
| (° C.) | | | | | | |
| Reaction | 2.1 | 2.1 | 2.1 | 2.1 | 2.1 | 2.1 |
| pressure (MPa) | | | | | | |
| W/F (g·h/mol) | 9 | 9 | 9 | 9 | 9 | 9 |

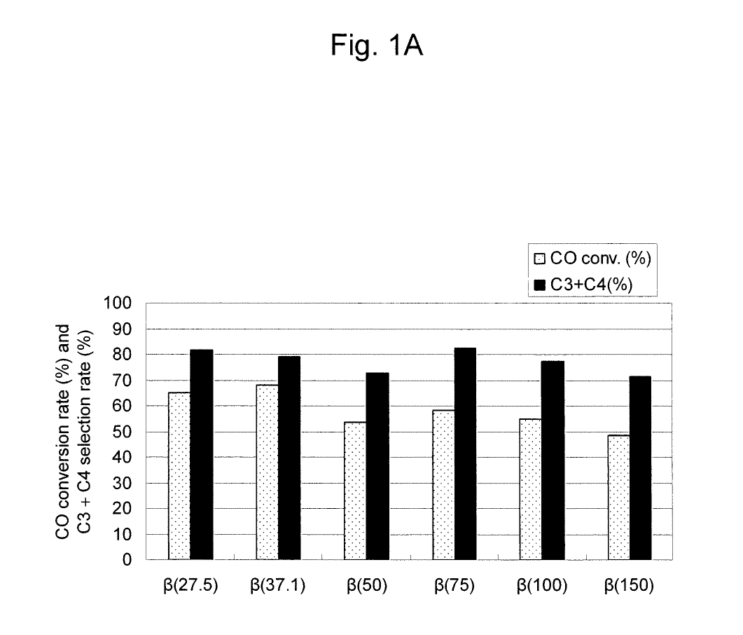

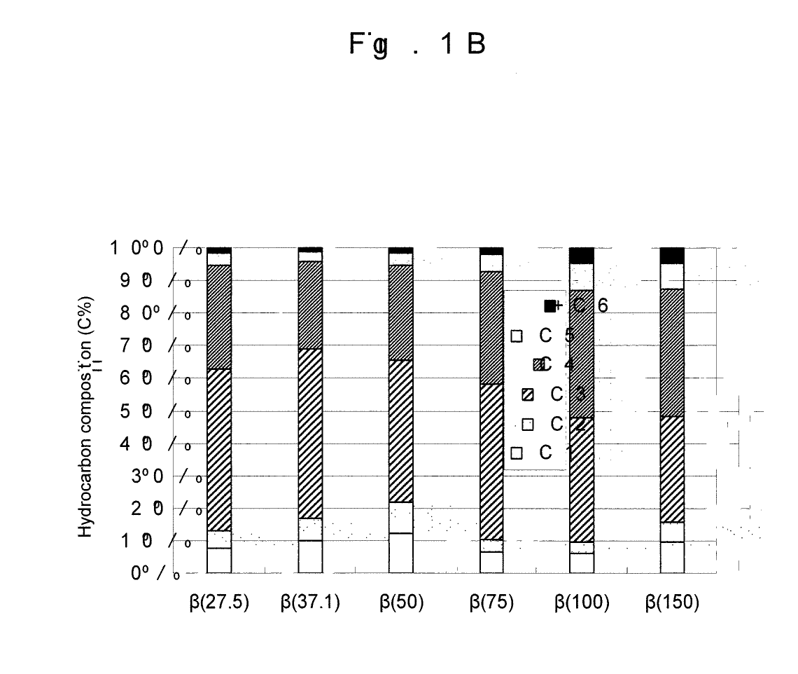

FIG. 1A shows the CO conversion rates and the C3+C4 selection rates obtained 3 hours after the initiation of the flow of a starting material gas. FIG. 1B shows the hydrocarbon compositions of products.

When a β-zeolite catalyst had an SiO₂:Al₂O₃ molar ratio of 37.1:1, the highest C3+C4 selection rate (i.e., the LPG selection rate) was obtained.

### Experiment 2

In this experiment, the relationship between the catalyst bed length (at the same catalyst amount) and the CO conversion rate (%), the C3+C4 selection rate (%), or the hydrocarbon composition (C %) of a product was examined.

Reaction conditions are as follows.

TABLE 3

| | Condition | Condition | Condition |
|---|---|---|---|
| Catalyst | 7 | 8 | 9 |
| Cu—Zn:Pd-β zeolite:Silica Q3 | 2:1:0 | 2:1:0 | 2:1:3 |
| (inactive component) (weight ratio) | | | |
| Loaded Pd content in Pd-β | 0.5 | 0.5 | 0.5 |
| zeolite (% by weight) | | | |
| SiO₂:Al₂O₃ (molar ratio) | 37.1:1 | 37.1:1 | 37.1:1 |
| in β-zeolite | | | |
| Catalyst weight (g) | 1 | 0.5 | 1 |
| Reaction temperature (° C.) | 350 | 350 | 350 |
| Reaction pressure (MPa) | 2.1 | 2.1 | 2.1 |
| W/F (g · h/mol) | 9 | 9 | 9 |

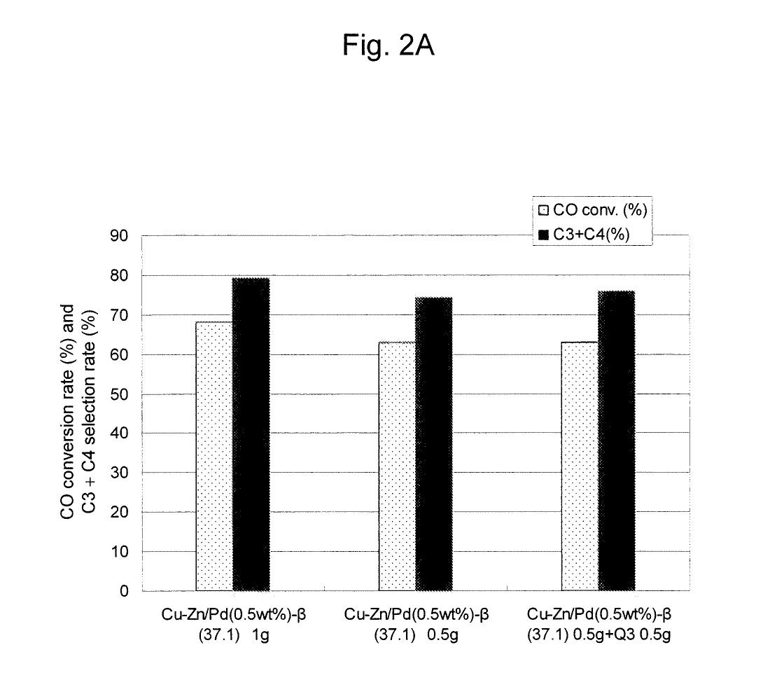

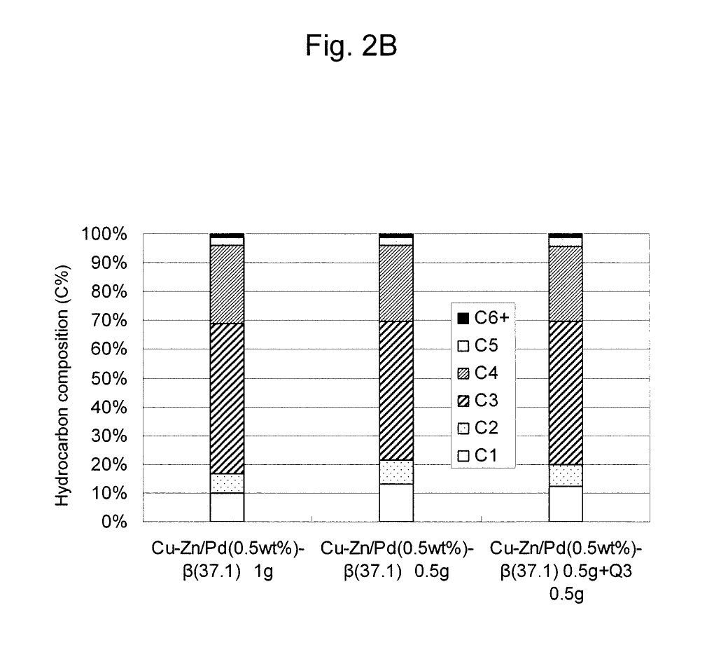

FIG. 2A shows the CO conversion rates and the C3+C4 selection rates obtained 3 hours after the initiation of the flow of a starting material gas. FIG. 2B shows the hydrocarbon compositions of products.

The longer the catalyst bed length, the better the CO conversion rate and the C3+C4 selection rate.

### Experiment 3

In this experiment, the relationship between the reaction temperature and the CO conversion rate (%), the C3+C4 selection rate (%), or the hydrocarbon composition (C %) of a product was examined.

Experimental conditions are as follows.

TABLE 4

| Catalyst | |
|---|---|
| Cu—Zn:Pd-β zeolite:Silica Q3 | 2:1:3 |
| (inactive component) (weight ratio) | |
| Loaded Pd content in Pd-β | 0.5 |
| zeolite (% by weight) | |
| SiO₂:Al₂O₃ (molar ratio) in β-zeolite | 37.1:1 |
| Catalyst weight (g) | 1 |
| Reaction temperature (° C.) | 300, 350, 375, 400 |
| Reaction pressure (MPa) | 2.1 |
| W/F (g · h/mol) | 2.3 |

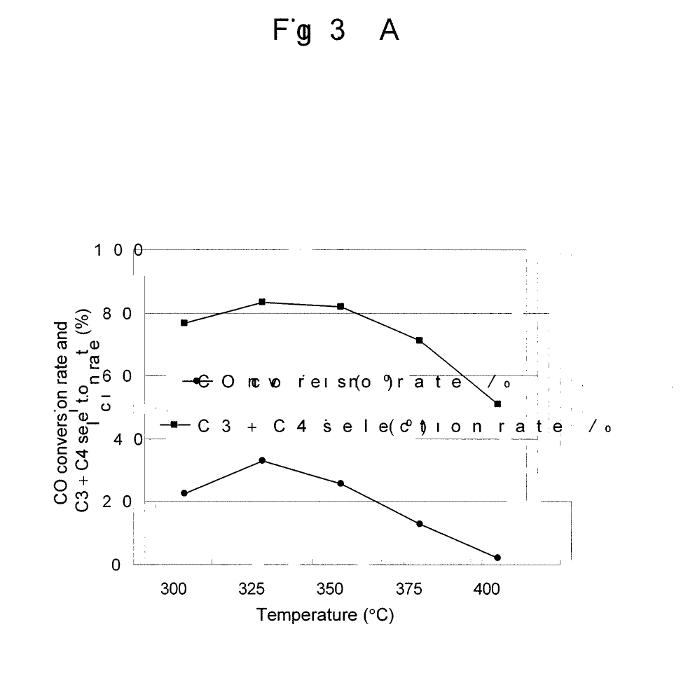

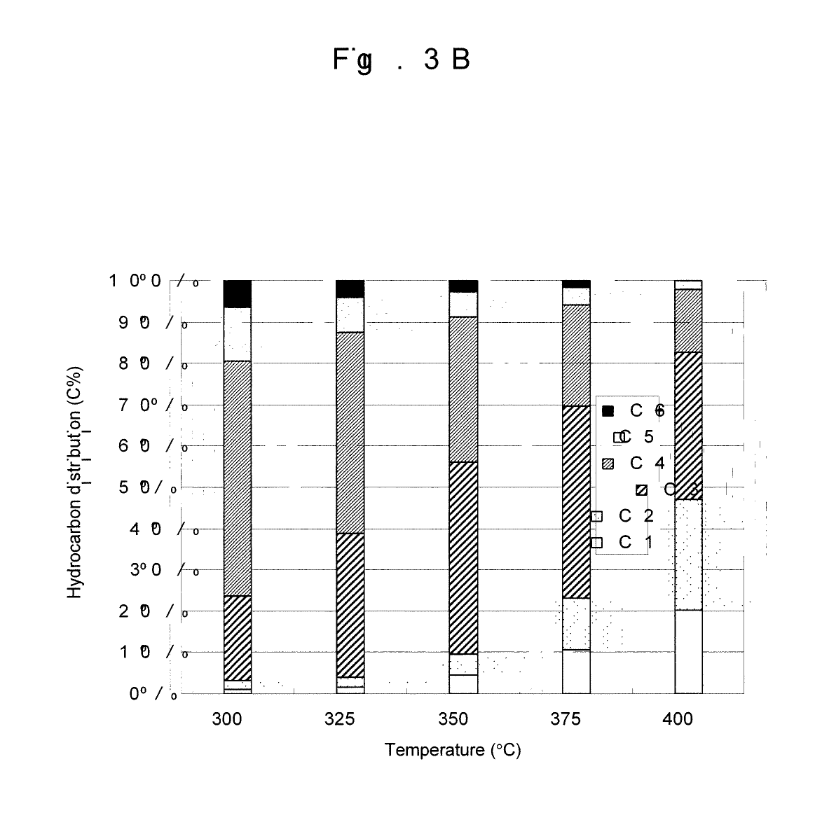

FIG. 3A shows the CO conversion rates and the C3+C4 selection rates obtained 3 hours after the initiation of the flow of a starting material gas. FIG. 3B shows the hydrocarbon compositions of products.

It was shown that the highest CO conversion rate and the highest C3+C4 selection rate were obtained when the reaction temperature was 325° C.

### Experiment 4

In this experiment, the relationship between the reaction pressure and the CO conversion rate (%), the C3+C4 selection rate (%), or the hydrocarbon composition (C %) of a product was examined.

Experimental conditions are as follows.

TABLE 5

| Catalyst | |
|---|---|
| Cu—Zn:Pd-β zeolite:Silica Q3 | 2:1:3 |
| (inactive component) (weight ratio) | |
| Loaded Pd content in Pd-β | 0.5 |
| zeolite (% by weight) | |
| SiO₂:Al₂O₃ (molar ratio) in β-zeolite | 37.1:1 |
| Catalyst weight (g) | 1 |
| Reaction temperature (° C.) | 350 |
| Reaction pressure (MPa) | 1.1, 1.6, 2.1, 3.1, 3.6 |
| W/F (g · h/mol) | 2.3 |

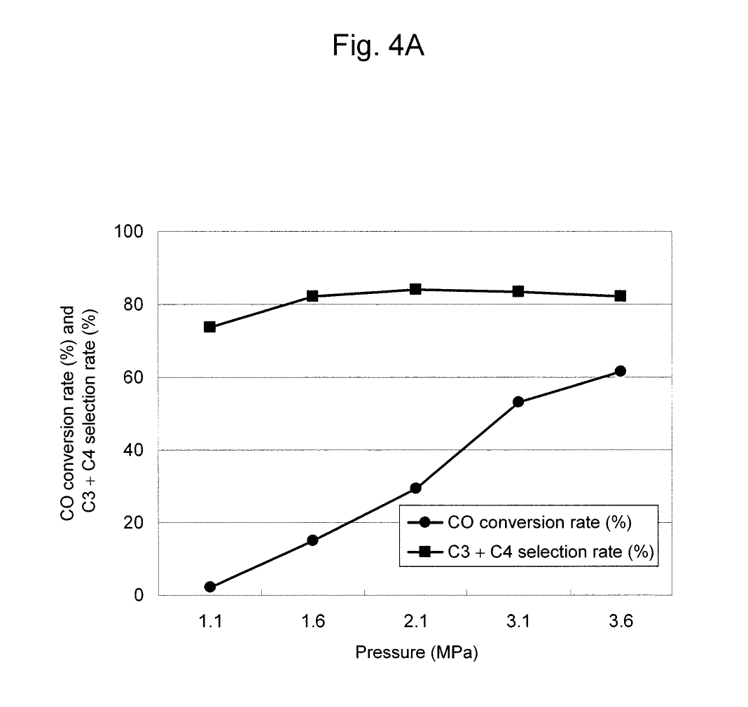

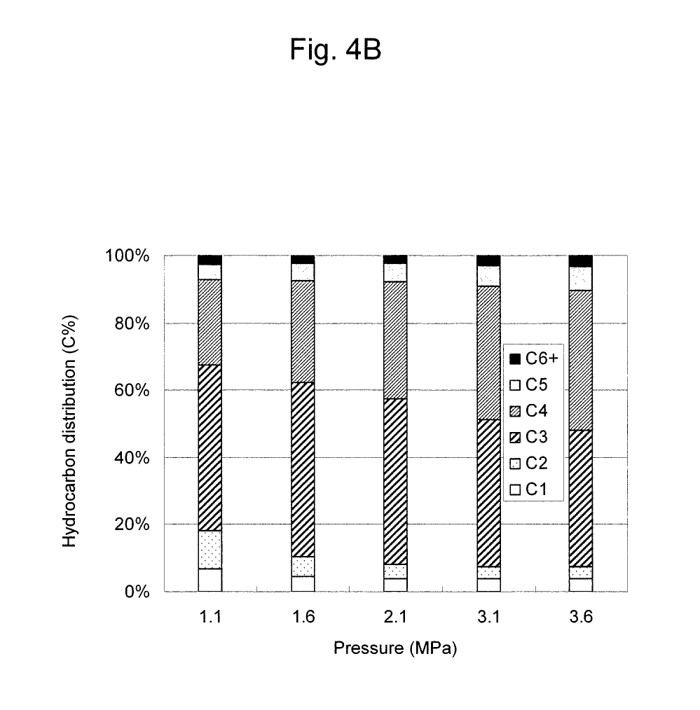

FIG. 4A shows the CO conversion rates and the C3+C4 selection rates obtained 3 hours after the initiation of the flow of a starting material gas. FIG. 4B shows the hydrocarbon compositions of product.

It was shown that the highest CO conversion rate was obtained when the reaction pressure was 2.1 MPa.

### Experiment 5

In this experiment, the relationship between the W/F value and the CO conversion rate (%), the C3+C4 selection rate (%), or the hydrocarbon composition of a product (C %) was examined.

Experimental conditions are as follows.

TABLE 6

| Catalyst | |
|---|---|
| Cu—Zn:Pd-β zeolite:Silica Q3 | 2:1:3 |
| (inactive component) (weight ratio) | |
| Loaded Pd content in Pd-β | 0.5 |
| zeolite (% by weight) | |
| SiO₂:Al₂O₃ (molar ratio) in β-zeolite | 37.1:1 |
| Catalyst weight (g) | 1 |
| Reaction temperature (° C.) | 350 |
| Reaction pressure (MPa) | 2.1 |
| W/F (g · h/mol) | 1.9, 2.3, 4.5, 9, 18 |

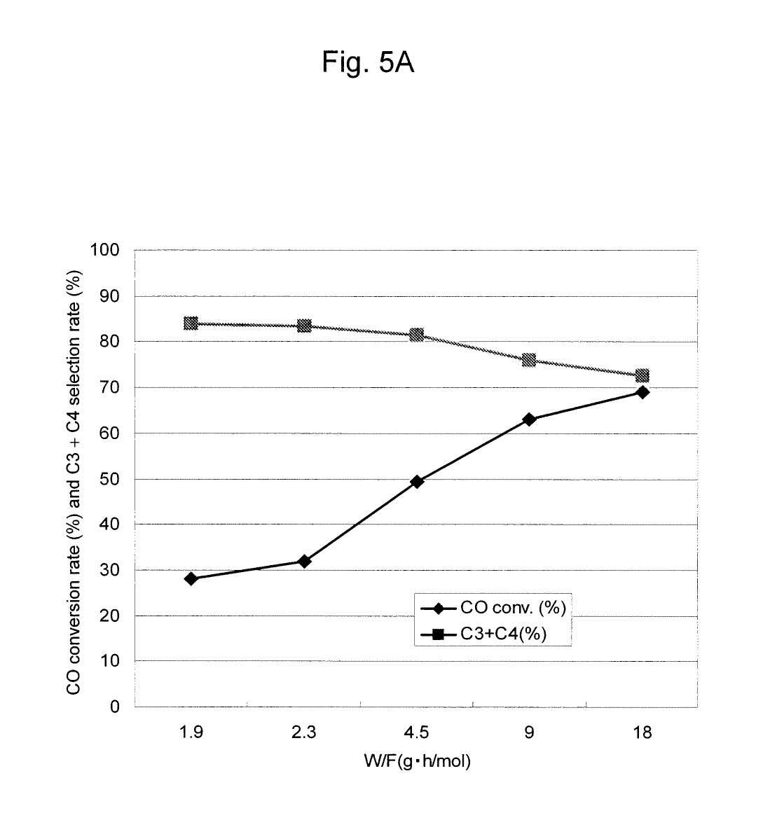

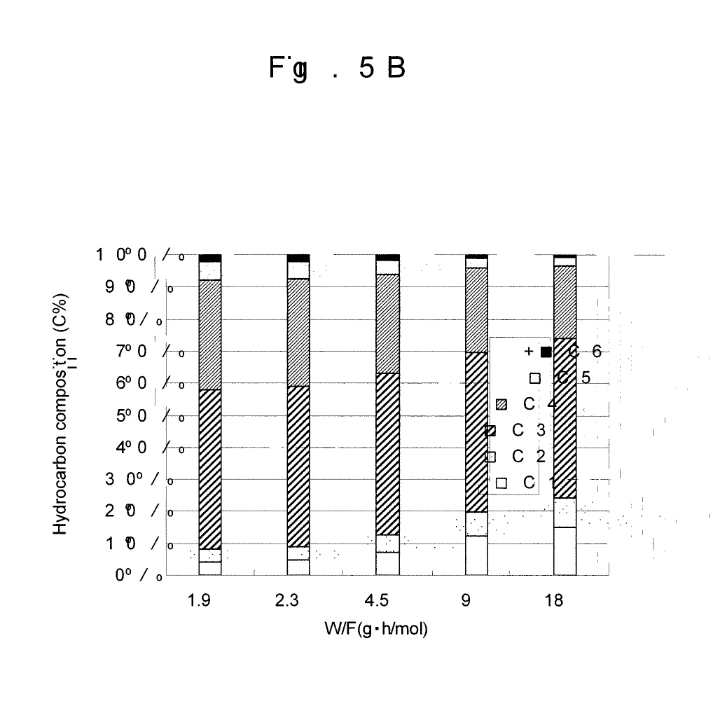

FIG. 5A. shows the CO conversion rates and the C3+C4 selection rates obtained 3 hours after the initiation of the flow of a starting material gas. FIG. 5B shows the hydrocarbon compositions of products.

When the W/F value was 1.9 g·h/mol, the highest C3+C4 selection rate was obtained.

### Experiment 6

In this experiment, the relationship between the loaded Pd content in β zeolite (% by weight) and time-dependent changes in the CO conversion rate (%), those in the C3+C4 selection rate (%), or those in the hydrocarbon composition (C %) of a product was examined.

TABLE 7

| | Condi- | Condi- | Condi- | Condi- |
|---|---|---|---|---|
| Catalyst | tion 10 | tion 11 | tion 12 | tion 13 |
| Cu—Zn:Pd-β zeolite:Silica Q3 | 2:1:0 | 2:1:0 | 2:1:0 | 2:1:0 |
| (inactive component) (weight ratio) | | | | |
| Loaded Pd content in Pd-β | 0 | 0.17 | 0.5 | 1 |
| zeolite (% by weight) | | | | |
| SiO₂:Al₂O₃ (molar ratio) in β- | 37.1:1 | 37.1:1 | 37.1:1 | 37.1:1 |
| zeolite | | | | |
| Catalyst weight (g) | 1 | 1 | 1 | 1 |
| Reaction temperature (° C.) | 350 | 350 | 350 | 350 |
| Reaction pressure (MPa) | 2.1 | 2.1 | 2.1 | 2.1 |
| W/F (g · h/mol) | 9 | 9 | 9 | 9 |

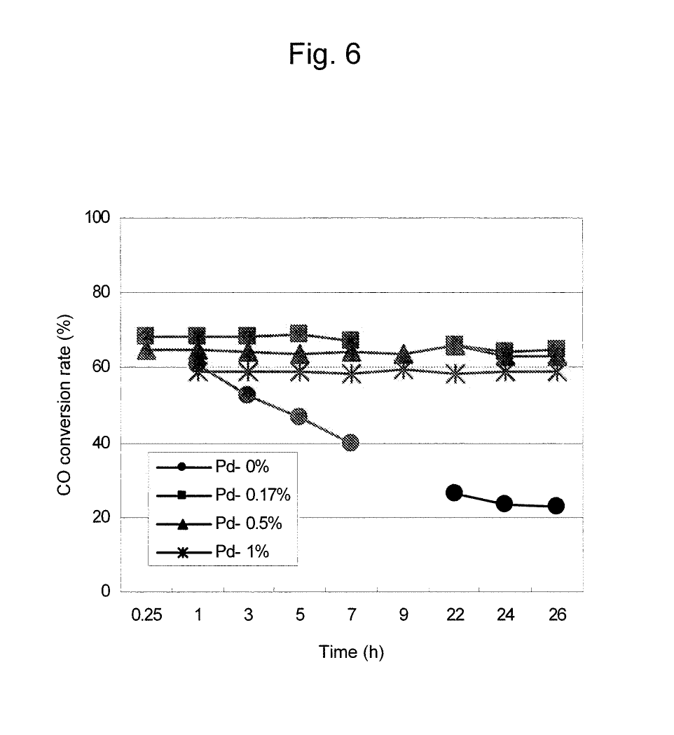

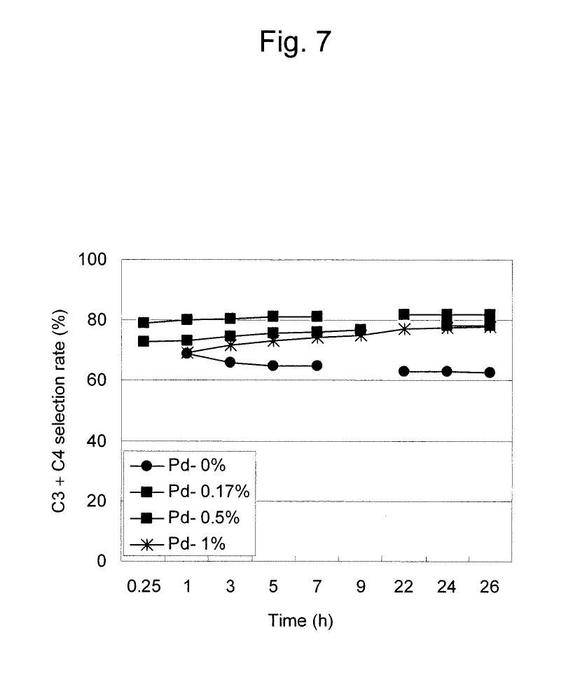

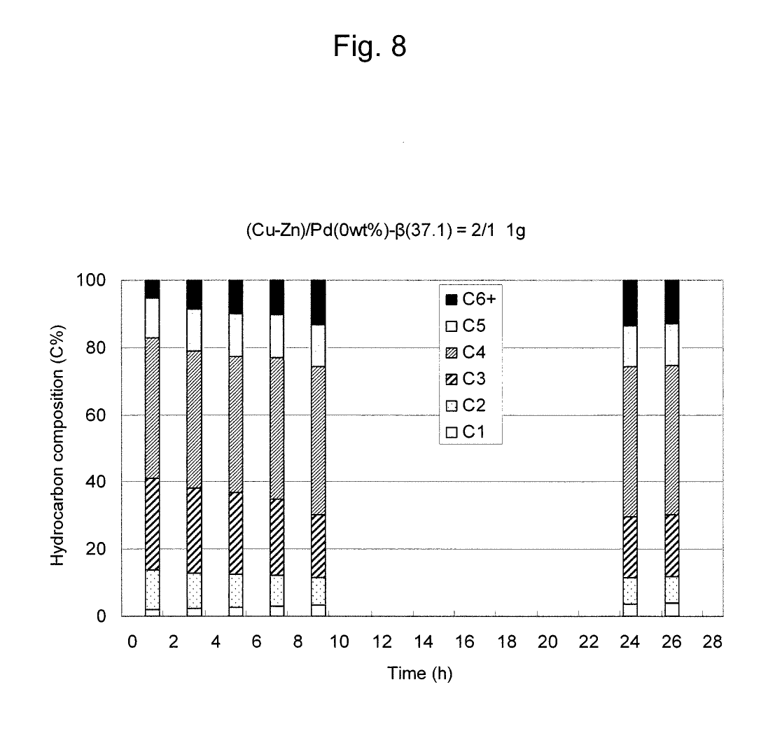

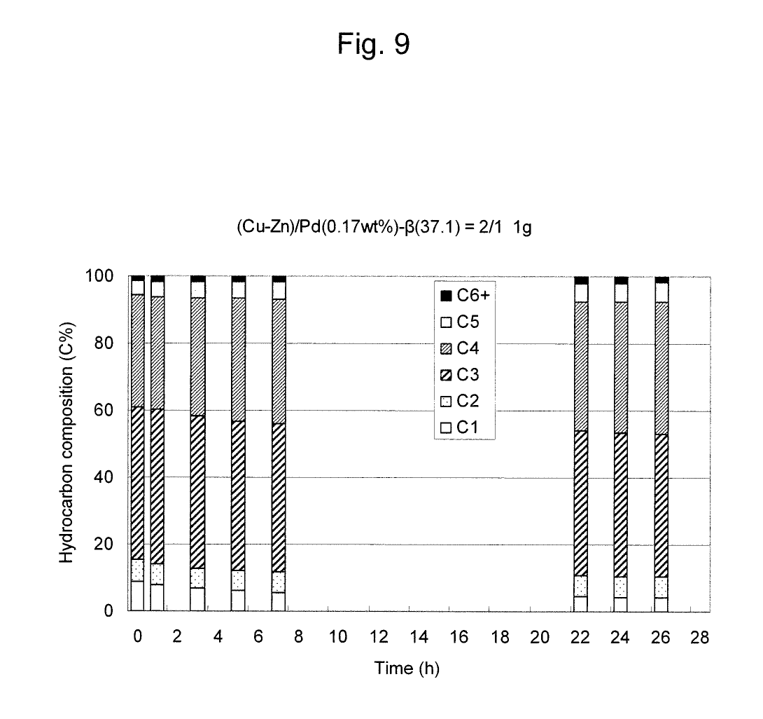

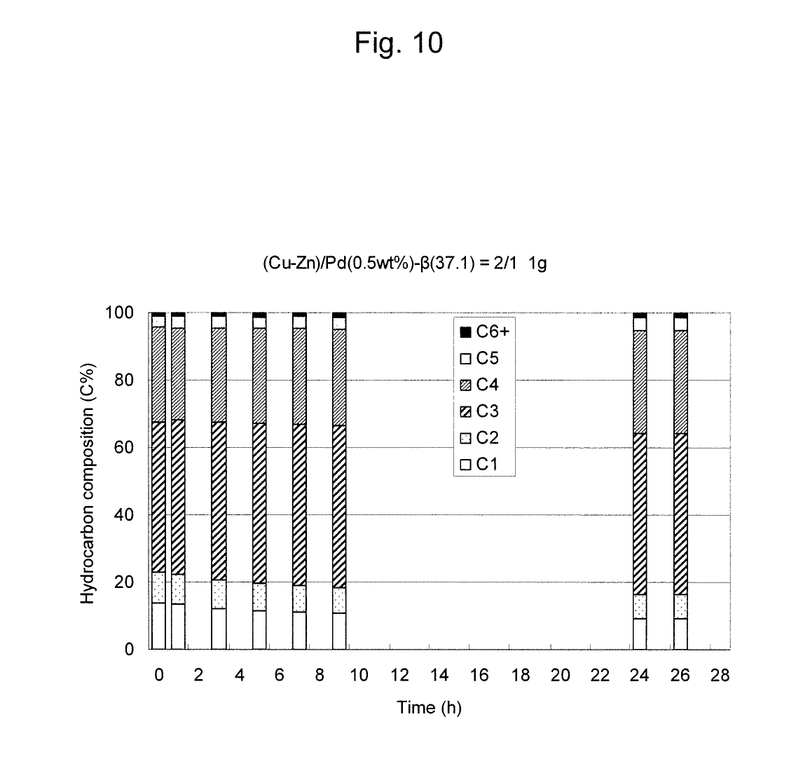

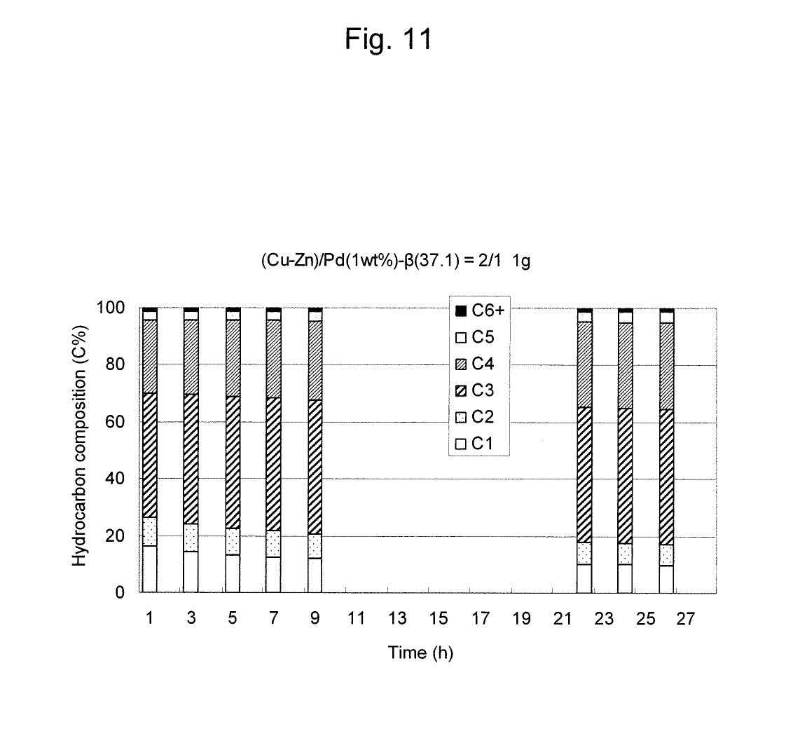

FIG. 6 shows time-dependent changes in the CO conversion rate in the cases of conditions 10 to 13. FIG. 7 shows time-dependent changes in the C3+C4 selection rate in the cases of conditions 10 to 13. FIG. 8 shows time-dependent changes in the hydrocarbon composition of a product in the case of condition 10. FIG. 9 shows time-dependent changes in the hydrocarbon composition of a product in the case of condition 11. FIG. 10 shows time-dependent changes in the hydrocarbon composition of a product in the case of condition 12. FIG. 11 shows time-dependent changes in the hydrocarbon composition of a product in the case of condition 13.

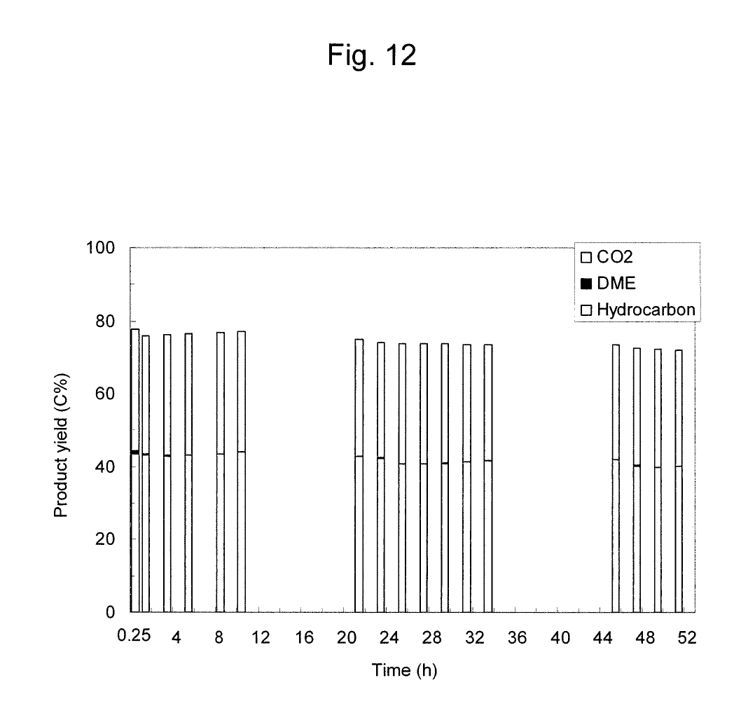

In addition, FIG. 12 shows time-dependent changes in the yields of CO₂, dimethyl ether (DME), and hydrocarbon of a product in the experiment based on condition 12. The terms containing the word “yield” used herein are defined as follows.

CO₂ yield(%)=[(CO₂ generation rate×1)/(inlet CO flow rate−outlet CO flow rate)]×CO conversion rate

DME yield(%)=[(DME generation rate×2)/(inlet CO flow rate−outlet CO flow rate)]×CO conversion rate

Hydrocarbon yield=[(C1 generation rate×1+C2 generation rate×2+C3 generation rate×3+C4 generation rate×4+C5 generation rate×5+C6 generation rate×6 . . . )/(inlet CO flow rate−outlet CO flow rate)]×CO conversion rate

In each equation, the units for “generation rate” are “mol/h” and the units for “flow rate” are “mol/h.”

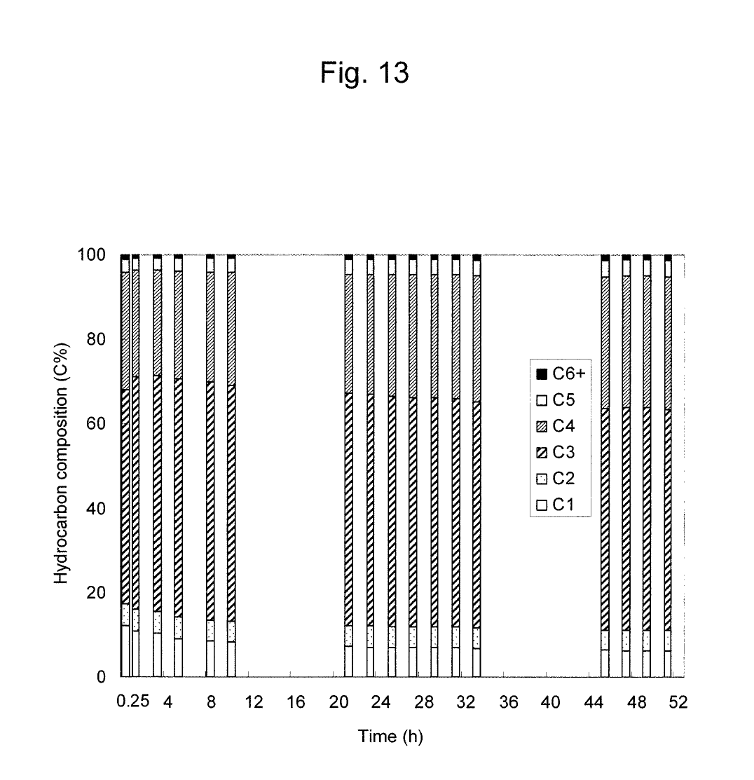

In addition, FIG. 13 shows time-dependent changes in the hydrocarbon composition (C %) of a product in the experiment based on condition 12 in greater detail than those shown in FIG. 10.

When the loaded Pd content was 0.17%, the highest CO conversion rate and the highest C3+C4 selection rate were obtained. In addition, when the loaded Pd content became high, a catalyst became more stable. Thus, even in the case involving loading of 0.5% Pd and in the case involving reaction for 52 hours, substantially no deterioration was observed in terms of activity. In the case in which no loading of Pd took place, the CO conversion rate and the C3+C4 selection rate tended to decrease over time.

### Experiment 7

In this experiment, the relationship between a method for loading Pd on β-zeolite (either an ion exchange method or an immersion method) and the product yield or the hydrocarbon composition of a product was examined.

Conditions are as follows.

TABLE 8

| | Condition 14 | Condition 15 |
|---|---|---|
| | Method for loading | |
| | Ion exchange | Immersion |
| Catalyst | method | method |
| Cu—Zn:Pd-β zeolite:Silica Q3 | 2:1:0 | 2:1:0 |
| (inactive component) (weight ratio) | | |
| Loaded Pd content in Pd-β | 0.5 | 0.5 |
| zeolite (% by weight) | | |
| SiO₂:Al₂O₃ (molar ratio) in β-zeolite | 27.5:1 | 27.5:1 |
| Catalyst weight (g) | 1 | 1 |
| Reaction temperature (° C.) | 350 | 350 |
| Reaction pressure (MPa) | 2.1 | 2.1 |
| W/F (g · h/mol) | 9 | 9 |

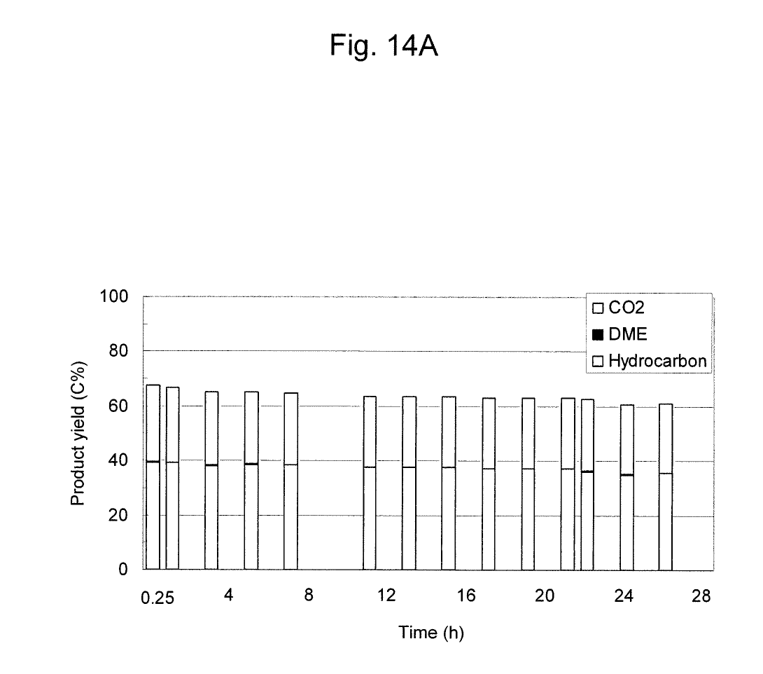

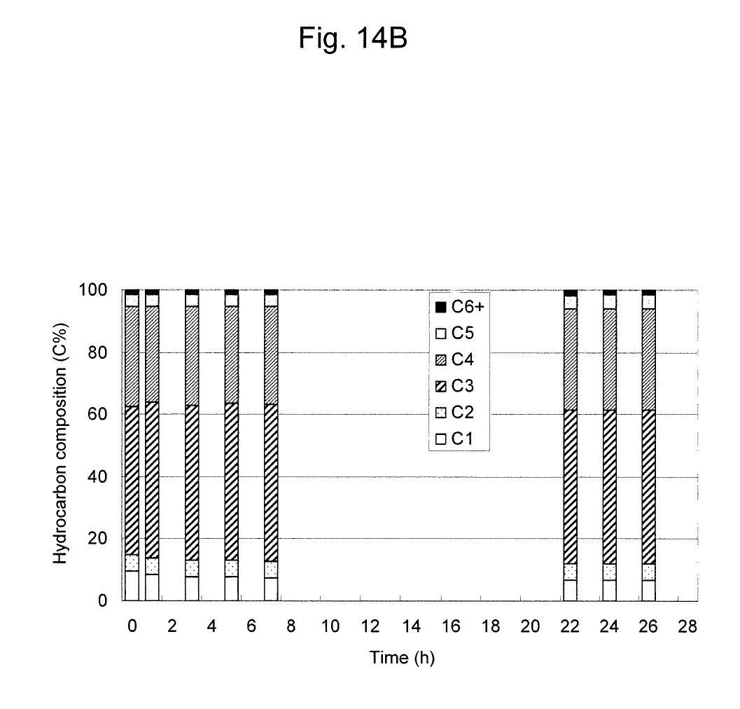

FIG. 14A shows time-dependent changes in the yields of CO₂, dimethyl ether (DME), and hydrocarbon of a product in the experiment based on condition 14 (an ion exchange method). FIG. 14B shows time-dependent changes in the hydrocarbon composition (C %) of a product in the experiment based on condition 14.

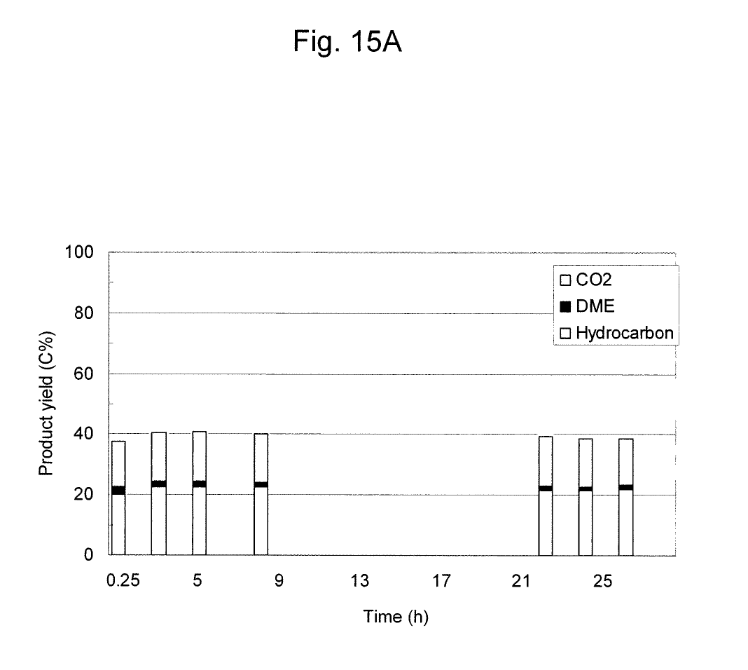

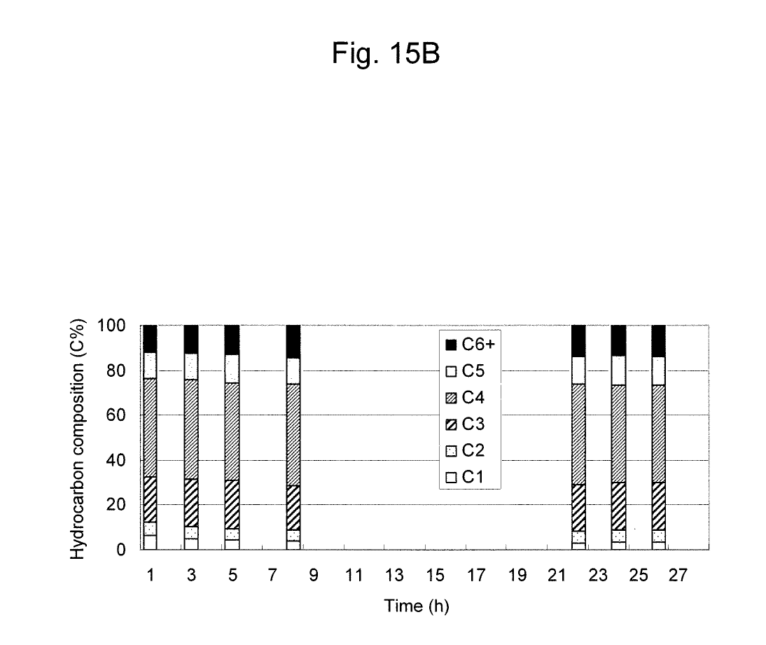

FIG. 15A shows time-dependent changes in the yields of CO₂, dimethyl ether (DME), and hydrocarbon of a product in the experiment based on condition 15 (an immersion method). FIG. 15B shows time-dependent changes in the hydrocarbon composition (C %) of a product in the experiment based on condition 15.

As shown in the figures, the hydrocarbon yield was increased to a greater extent with the use of the catalyst subjected to Pd loading by the ion exchange method than with the catalyst subjected to Pd loading by the immersion method. Also, the LPG selectivity (C3+C4 selectivity) was increased.

### Example 2

In this Example, LPG synthesis reaction was carried out with the use of a starting material gas in the same manner as in the case of Example 1, except that conditions listed in table 9 were employed as reaction conditions and that the following procedures were implemented as experimental operational procedures instead of the procedures described in item 7 in Example 1.

### Experimental Operational Procedures in this Example

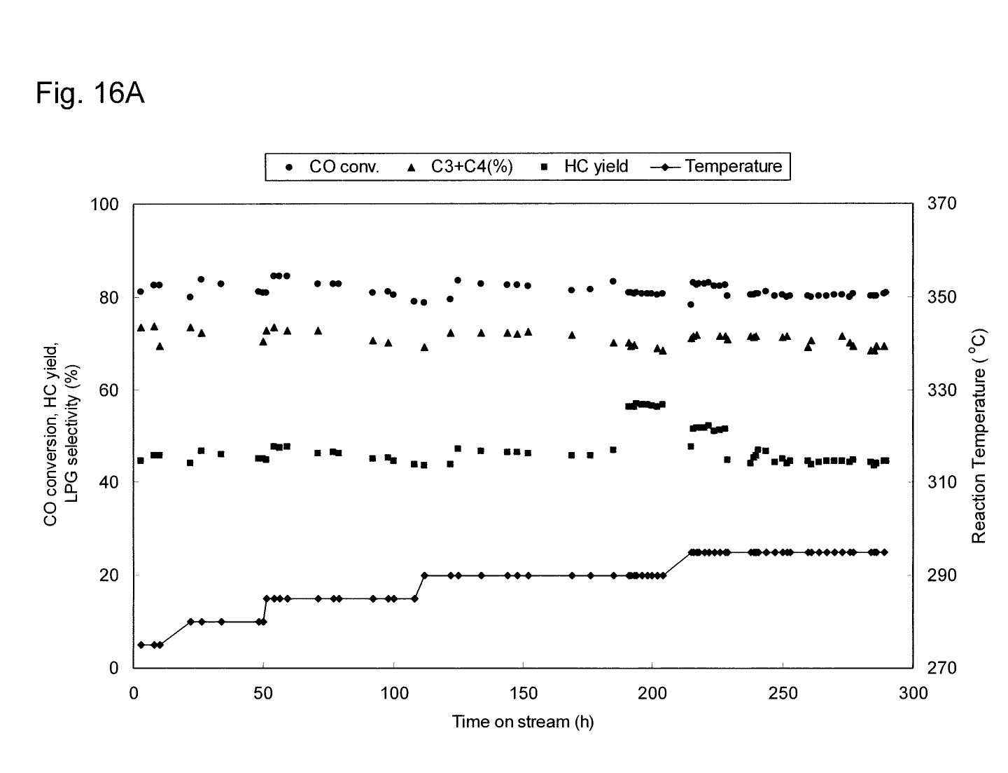

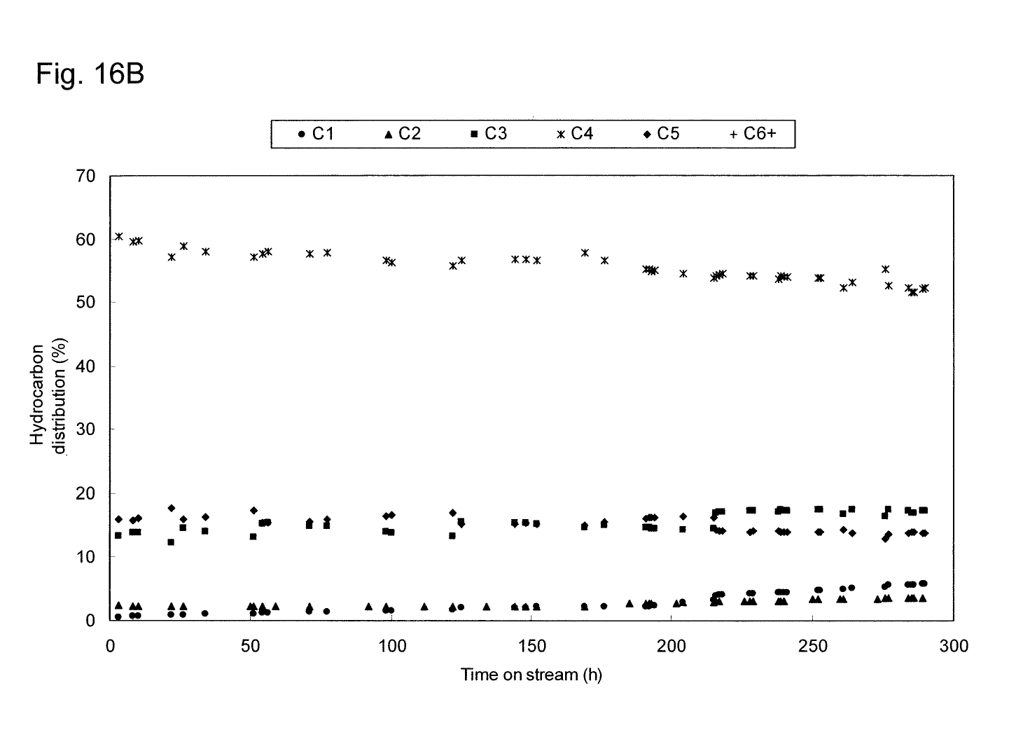

(1) A reaction tube is filled with glass wool, glass beads, a catalyst, and glass beads in such order and placed in a furnace.
(2) Leakage from a reactor is checked with a flow of N₂ at 100 ml/min.
(3) A flow of N₂ at 100 ml/min is supplied to the reactor and the temperature is increased to 250° C.
(4) When the temperature reaches 250° C., drying is carried out for 2 hr while the temperature is maintained.
(5) A flow of high purity H₂ at 5 ml/min is supplied with the flow of N₂, the flow of N₂ is set at 95 ml/min, and the temperature is increased to 300° C.
(6) When the temperature reaches 300° C., reduction is carried out for 3 hr while the temperature is maintained.
(7) The supply of N₂ and H₂ is terminated, a mixed gas (CO: CO₂: H₂: Ar=28.4:4.1:64.4:3.1 (molar ratio)) is supplied, and the pressure is increased to 2.1 MPa.
(8) The temperature is increased to a temperature at which a reaction is initiated.
(9) Reaction is initiated.
(10) Sampling is carried out at regular time intervals and the generated gas is analyzed.
(11) The temperature is controlled such that the CO conversion rate is maintained at approximately 80% (the temperature is increased in a stepwise manner as shown in FIG. 16A).
(12) The supply of the reaction gas is terminated, the reaction is completed, and the temperature is decreased in the presence of a flow of the reaction gas.

TABLE 9

| Catalyst | |
|---|---|
| Cu—Zn:Pd-β zeolite:Silica Q3 | 0.95:1:0 |
| (inactive component) (weight ratio) | |
| Loaded Pd content in Pd-β | 0.5 |
| zeolite (% by weight) | |
| SiO₂:Al₂O₃ (molar ratio) in β-zeolite | 37.1:1 |
| Catalyst weight (g) | 1 |
| Reaction temperature (° C.) | 270-295 |
| Reaction pressure (MPa) | 2.1 |
| W/F (g · h/mol) | 8.9 |

The reaction was carried out for approximately 300 hours. Sampling of a generated gas was carried out in a time-dependent manner FIG. 16A shows the CO conversion rate (%), the hydrocarbon yield (%), and the LPG selectivity (C3+C4 selectivity) (%) at each time point after the initiation of the reaction. FIG. 16B shows the product composition with regard to each hydrocarbon (C %) at each time point after the initiation of the reaction.

As a result of the above experiments, it has been revealed that the catalyst of the present invention exhibits good activity (e.g., C3+C4 selectivity) for a long period of time (approximately 300 hours). That is, the catalyst of the present invention is sufficiently durable for industrial use.

All publications, patents, and patent applications cited herein are incorporated herein by reference in their entirety.

1. A catalyst for producing a liquefied petroleum gas mainly consisting of propane or butane by reacting carbon monoxide with hydrogen, the catalyst comprising a Cu—Zn-based catalyst component and a β-zeolite catalyst component loaded with Pd.

2. The catalyst according to claim 1, wherein the loaded Pd content in the β-zeolite catalyst component loaded with Pd is 0.1% to 1% by weight.

3. The catalyst according to claim 1, wherein β-zeolite in the β-zeolite catalyst component loaded with Pd is β-zeolite having an SiO₂:Al₂O₃ molar ratio of 10:1 to 150:1.

4. The catalyst according to claim 1, wherein the weight ratio of the Cu—Zn-based catalyst component to the β-zeolite catalyst component loaded with Pd is 4:1 to 1:4.

5. The catalyst according to claim 1, wherein the β-zeolite catalyst component loaded with Pd is prepared by a method comprising the steps of immersing β-zeolite in a solution containing Pd(NH₃)₄Cl₂ and removing Cl from the β-zeolite treated in the previous step.

6. A method for liquefied petroleum gas production, comprising reacting carbon monoxide with hydrogen in the presence of the catalyst according to claim 1 so as to produce a liquefied petroleum gas mainly consisting of propane or butane.

7. The method according to claim 6, wherein the reaction temperature for reacting carbon monoxide with hydrogen is 290° C. to 375° C.

8. The method according to claim 6, wherein the reaction pressure for reacting carbon monoxide with hydrogen is 2 to 5 MPa.

9. The method according to claim 6, wherein a value obtained by dividing the amount of a catalyst used for reacting carbon monoxide with hydrogen (units: “g”) by the inlet gas flow rate (units: “mol/h”) is 1.9 to 18 g·h/mol.

10. A method for liquefied petroleum gas production, comprising a liquefied petroleum gas production step of allowing a synthesis gas to flow through a catalyst layer containing the catalyst according to claim 1 so as to produce a liquefied petroleum gas mainly consisting of propane or butane.

11. A method for liquefied petroleum gas production, comprising:
(1): a step of producing a synthesis gas with the use of a carbon-containing starting material and at least one member selected from the group consisting of H₂O, O₂, and CO₂; and
(2): a liquefied petroleum gas production step of allowing the synthesis gas to flow through a catalyst layer containing the catalyst according to claim 1 so as to produce a liquefied petroleum gas mainly consisting of propane or butane by.
